(19) United States
(12) Patent Application Publication
(10) Pub. No.: US 2013/0053801 A1
(43) Pub. Date: Feb. 28, 2013

(54) MECHANICAL LEG BAG RELEASE APPARATUS AND METHOD

(71) Applicant: James W. Thomas, Windsor Heights, IA (US)

(72) Inventor: James W. Thomas, Windsor Heights, IA (US)

(21) Appl. No.: 13/218,616

(22) Filed: Aug. 26, 2011

(51) Int. Cl. A61F 5/44

(52) U.S. Cl. 604/323; 604/327

(57) ABSTRACT

A leg bag release system is presented having a block member with a drain tube channel and a crimping member slidably received within the block member. A cable assembly connects the block member to a lever assembly. When the lever assembly is activated, the crimping member is withdrawn from the drain tube channel allowing a patient to empty a leg bag without the assistance of another person.

### FIELD OF THE INVENTION

This invention relates to a leg release. More specifically, and without limitation, this invention relates to a mechanical leg bag release.

### BACKGROUND OF THE INVENTION

Leg bags are well known in the art. Many devices and methods have been developed for emptying leg bags. Conventionally, a patient who is bound to a wheelchair wears a leg bag which collects fluid. When this leg bag fills, a nurse, orderly or assistance provider is required to help the patient empty the leg bag. This process is labor intensive, expensive, and requires the presence of another person who is often a paid assistance provider.

To improve upon this manual and labor intensive process, electric powered release systems were developed. While these devices eliminated the need for assistance, they are expensive and require electric power. In addition, these systems rely upon an electrical solenoid which often burns-out requiring replacement. Also, these devices often have complicated and intricate pieces which require daily cleaning or disposal. Accordingly, there exists a need in the art for a device that addresses these deficiencies.

Therefore, an object of the present invention is to provide a method and apparatus for emptying a leg bag that does not require the assistance of another person.

Another object of the present invention is to provide a leg bag release that is less expensive does not require electric power.

Yet another object of the present invention is to provide a leg bag release that does not require manual labor assistance of another person to empty the leg bag.

These and other objects, features, or advantages of the present invention will become apparent to one of ordinary skill in the art from the specification and claims.

### SUMMARY OF THE INVENTION

A leg bag release system having a block member with a drain tube channel and a crimping member sidably received within the block member. A cable assembly connects the block member to a lever assembly. When the lever assembly is activated, the crimping member is withdrawn from the drain tube channel allowing a patient to empty a leg bag without the assistance of another person.

### BRIEF DESCRIPTION OF THE DRAWINGS

FIG. 1 is a perspective view of the device connected to a person and a wheel chair;

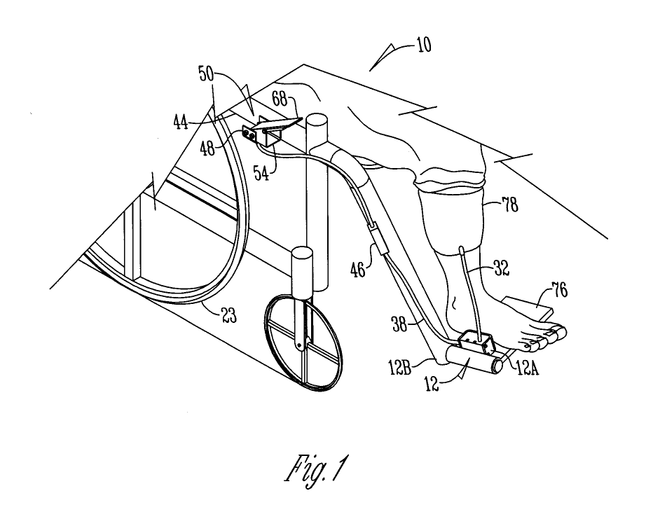

FIG. 2 is a perspective view of the exploded block assembly; and

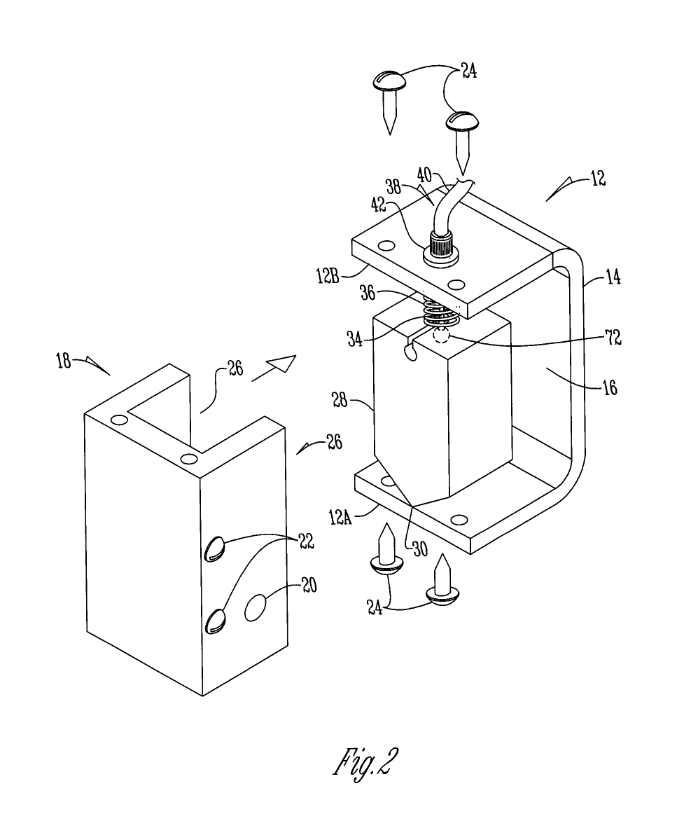

FIG. 3 is a perspective view of a release assembly.

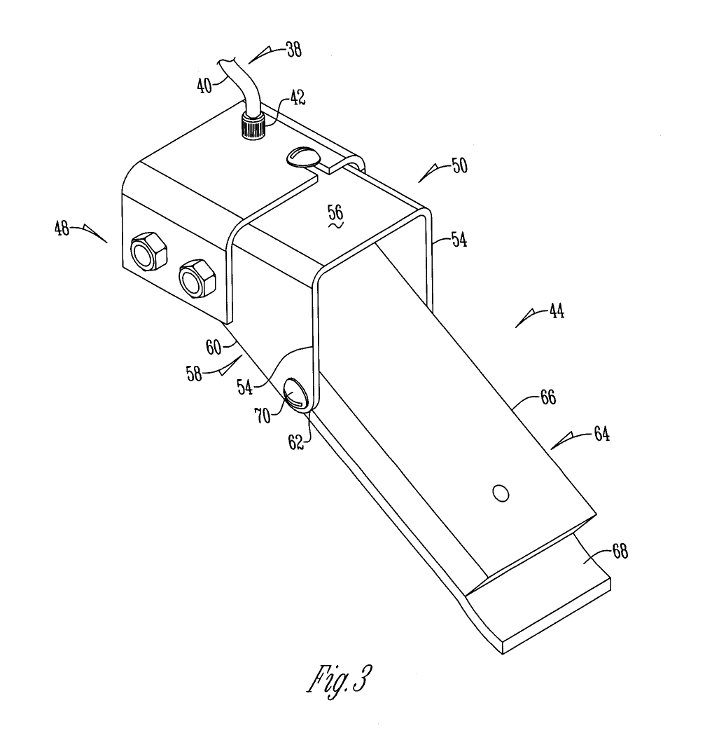

### DETAILED DESCRIPTION OF THE PREFERRED EMBODIMENT

With reference to the drawings, a leg bag release system 10 has block assembly 12 having a first end 12A and a second end 12B. The block assembly 12 has a block bracket 14 which preferably has a C-shape, a U-Shape or a rectangular shape which defines an open interior space or channel 16. In a preferred embodiment, block bracket 14 is made of a high-grade stainless steel material so as to provide rigidity, longevity and reduce the need for cleaning. However, any other material which meets these requirements is hereby contemplated such as plastic, composite or the like materials.

Matingly received by and positioned within the interior space or channel 16 of block bracket 14 is block member 18. Positioned adjacent the first end 12A of block member 18 is drain tube channel 20. Preferably drain tube channel 20 is a cylindrically-shaped hole, bore or channel which passes through the entire body of block member 18 from its top side through to its bottom side. Block member 18 also has at least one, and preferably two, connecting bolts 22 which also pass through the body of block member 18 from its top side through to its bottom side. These bolts 22 are used to connect block member 18 to a wheelchair 23 or other object. Alternatively, bolts 22 are connected to block member 18 in any other manner, or alternately bolts 22 are connected to block bracket 14.

Block bracket 14 and block member 18 are connected to one another by passing a screw or bolt 24 through block bracket 14 an into block member 18 at each of their first and second ends 12A, 12B. Alternatively any other means known in the art are used to connect the two pieces together.

Block member 18 has an open interior cavity 26 which extends from its first end 12A to its second end 12B. This interior cavity 26 intersects or cuts across drain tube channel 20. Positioned within interior cavity 26 is crimping member 28. Preferably crimping member 28 is matingly and slidably received within interior cavity 26 with close tolerances so as to guide its sliding. The first end 12A of crimping member 28 has a square, rounded or pointed nose 30 so as to effectively crimp a drain tube 32 positioned within and passing through drain tube channel 20. The second end 12B of crimping member 28 is connected to a release cable 34 to effectuate its actuation.

Also positioned within interior cavity 26, between the second end 12B of crimping member 28 and block bracket 14, is crimping spring 36. Crimping spring 36 acts to push nose 30 of crimping member 28 towards the first end 12A of block assembly 12 and across drain tube channel 20. Preferably, block member 18 and crimping member 28 are made of a durable plastic, composite or ultra high molecular weight material that is easily machined and easily cleaned. However, any other durable rigid and easily cleaned material is hereby contemplated

Connected to the second end 12B of block assembly 12 is cable assembly 38. Cable assembly 38 has an outer sheath 40 through which cable 34 passes. Cable assembly 38 is connected at its first end to the second end 12B of block bracket 14 by way of a threaded adjustable collar assembly 42. The second end of cable assembly 38 is connected to release assembly 44 also by way of a threaded adjustable collar assembly 42. Positioned between the first end and second end of cable assembly 38 is tumbler 46. Tumbler 46 rotatably adjusts to extend and retract the length of outer sheath 40 so as to adjust the length and tension of cable assembly 38 so as to properly affect actuation of the leg bag release system 10. The threaded adjustable collar assembly 42 at each end of cable assembly 38 also adjust the length and tension of cable assembly 38.

Release assembly 44 has a first mounting bracket 48 connected to a second mounting bracket 50. First mounting bracket 48 is of any size and shape needed to connect release assembly to a wheelchair 23 or other object. Preferably, first mounting bracket 48 is L-shaped, however any other shape his hereby contemplated. Second mounting bracket 50 extends upwardly from the first mounting bracket 48. Preferably second mounting bracket 50 has a base 54 from which a pair of sidewalls 56 extend in parallel spaced relation. Opposite base 54, sidewalls 56 terminate at a top edge 58 having a flat angled portion 60 which extends upwardly to an arcuate cam surface 62. Rotatably positioned between sidewalls 56 is lever 64.

Lever 64 has a body 66 and a cover 68. Lever 64 is pivotally connected to sidewalls 54 by way of shaft 70 which passes through sidewalls 54 and body 66 of lever 64. Preferably shaft 70 is positioned adjacent and at the center of the arc of the arcuate cam surface 62 and acts as a pivot point for lever 64 so as to actuate block assembly 12. Preferably the second end of cable assembly 38 connects to first mounting bracket 48 by way of adjustable collar assembly 42. Cable 34 then extends past the end of outer sheath 40 and through the first mounting bracket 48 and second mounting bracket 50. Cable 34 then connects to the body 66 of lever arm 64 by way of bulbous end 72 fitting within key slot 74. Similarly, the bulbous end 72 of cable 34 connects to key slot 74 in crimping member 28.

To install the leg bag release system 10, first mounting bracket 48 is connected to wheelchair 23 at any convenient location where a patient can actuate the leg bag release system 10 without the assistance of another person. Next, block assembly 12 is connected to a foot-pad 76 of wheelchair 23. This is accomplished by way of bolting block assembly 12 to foot-pad 76 through the use of bolts 22. A hole of adequate size is then drilled through foot-pad 76 in alignment with drain tube channel 20. Next the leg bag 78 is attached to the patient's leg and drain tube 32 is threaded through drain tube channel 20 of block assembly 12, through the hole in foot-pad 76 and out the bottom of foot-pad 76.

In operation, in a static position, crimping spring 36 applies a force against crimping member 28 such that crimping member 28 engages and closes or crimps drain tube 32. In this static position, crimping member extends across drain tube channel 20, and lever 64 lies flush against the flat angled portion 60 of second mounting bracket 50. In this way, when in an un-activated static position, leg bag release system 10 prevents fluids from escaping the leg bag 78.

When patient's leg bag 78 needs to be emptied, the patient positions their wheelchair 23 such that the foot-pad 76 having the drain tube 32 extending therethrough is positioned above a drain. Next, the patient activates the leg bag release system 10 by grasping lever 64 and rotating lever 64 on shaft 70. When doing so, lever 64 lifts away from the flat angled portion 60 of second mounting bracket 50 and rotates on the arcuate cam surface 62 of second mounting bracket 50. This movement pulls cable 34 through outer sheath 40 thereby pulling crimping member 28 within block member 12 and away from drain tube channel 20. In this way fluids are allowed to escape from leg bag 78.

From the above discussion it will be appreciated that a patient can now empty their own leg bag 78 without assistance. Also, as the leg bag release system 10 merely engages the drain tube 32, no portion of the system 10 becomes soiled and therefore daily cleaning is no longer required. Accordingly, the leg bag release system provides and offers many advantages over the prior art. It will be appreciated by those skilled in the art that other various modifications could be made to the device without parting from the spirit and scope of this invention. All such modifications and changes fall within the scope of the claims and are intended to be covered thereby.

1. A leg bag release system comprising:
a block member having a drain tube channel;
a crimping member slidably received within the block member;
a cable assembly connected at its first end to the crimping member;
the cable assembly connected at its second end to a lever assembly; and
wherein when the lever assembly is actuated the crimping member slides within the block member so as to drain a leg bag.

2. The system of claim 1 wherein when in a static position the crimping member extends across the drain tube channel.

3. The system of claim 1 wherein when actuated the crimping member is withdrawn from the drain tube channel.

4. The system of claim 1 further comprising a spring positioned between the drain tube channel and the crimping member.

5. The system of claim 1 wherein the lever assembly is connected to a wheelchair.

6. The system of claim 1 wherein the block member is connected to a foot pad of a wheelchair.

7. The system of claim 1 wherein a drain tube passes through the drain tube channel.

8. The system of claim 1 wherein the cable assembly has a tumbler so as to adjust the cable assembly.

9. A method of using a leg bag release system comprising:
obtaining a leg bag release system comprising a block member having a drain tube channel, a crimping member slidably received within the block member, and a cable assembly connecting the crimping member to an activation member; and
passing a drain tube connected to the leg bag through the drain tube channel such that when the leg bag release system is in an un-activated state the crimping member engages the drain tube preventing fluids from passing through the drain tube.

10. The method of claim 10 wherein when the leg bag release system is activated the crimping member withdraws from the drain tube channel allowing fluids to pass through the drain tube.
